(19) United States
(12) Patent Application Publication
(10) Pub. No.: US 2019/0262191 A1
(43) Pub. Date: Aug. 29, 2019

(54) Smart diaper having pre-treated material for accurate sensing of a soiled area

(71) Applicant: Gallop Creation USA Inc., Milpitas, CA (US)

(72) Inventors: Li Li, Femont, CA (US); Zhigang Wang, Fremont, CA (US)

(21) Appl. No.: 15/905,812

(22) Filed: Feb. 26, 2018

(51) Int. Cl. A61F 13/42; A61F 13/49; A61F 13/53; A61F 13/44

(52) U.S. Cl. CPC A61F 13/42; A61F 13/49; A61F 2013/424; A61F 13/44; A61F 13/53

(57) ABSTRACT

A smart diaper to be worn by a user, includes an absorption pad that can absorb a liquid to form a soiled area, a first ionic compound in the absorption pad, a pair of electrode sensing circuits that includes a first electrode sensing circuit and a second electrode sensing circuit, which are in contact with the absorbent material, and a controller that can apply a voltage across the pair of electrode sensing circuits and to produce an electrical current that increases with area of the soiled area, wherein the first ionic compound can release ions in the soil area to increase the electrical current.

### BACKGROUND OF THE INVENTION

The present application relates to a smart diaper that can sense whether a diaper has been soiled, and in particular, to a smart diaper having improved sensing accuracy of the soiled area.

There has been a long-felt need for the detection and real-time notifications of soiled diapers for about soiled diapers so that they can be promptly changed by caregivers. Diaper soil detection and alert are important for babies, who could develop diaper rash when left wearing soiled diapers for long periods of time, as well as for incontinent adults, who are unable to communicate with attendants and are susceptible to infections caused by wet diapers.

For cost reasons, many smart diaper products rely on reusable sensors. The caregiver needs to remove and to re-attach a reusable sensor during each diaper change. A smart diaper can also be embedded with a disposable sensor, which simplifies the diaper change process and achieves a better user experience. The cost of a single-use smart diaper must be sufficiently low to be accepted by consumers.

For smart diapers having reusable or single-use sensors, however, there is still a need to improve detection sensitivity and accuracy once soiling occurs in the diapers.

### SUMMARY OF THE INVENTION

The presently application discloses a smart diaper that can more timely and more accurately sense the wet areas therein. The disclosed smart diaper is embedded with a conductivity sensor that can measure electrical resistance variations of the materials between conductive electrodes. In particular, the materials between the conductive electrodes are pre-treated to enhance electrical resistance variations between a dry state and a soiled state in the diaper.

One or more materials can be pre-treated with ionic compounds in the disclosed smart diaper to improve detection sensitivity. The one or more materials pre-treated are located at different positions in a diaper structure; their treatments can not only vary the timing of release of the ionic compounds, but also help the identifying the locations of soiling areas in the disclosed diaper.

Moreover, by improving signal-to-noise ratio of soil detection, the disclosed smart diaper can overcome the problem of false alarms in conventional technologies by probably setting detection threshold.

Furthermore, the sensor in the disclosed smart diaper can be connected to a controller that can detect wetting of the diaper from measured signals, computing wet area, and transmitting a (wireless) signal to an external device such as a smart phone to alert caretakers. The controller can also send signal to a server, which can send a message to alert a caregiver. The measurements can be performed without affecting users' normal daily activities.

In one general aspect, the present invention relates to a smart diaper to be worn by a user, includes an absorption pad that can absorb a liquid to form a soiled area, a first ionic compound in the absorption pad, a pair of electrode sensing circuits that includes a first electrode sensing circuit and a second electrode sensing circuit, which are in contact with the absorbent material, and a controller that can apply a voltage across the pair of electrode sensing circuits and to produce an electrical current that increases with area of the soiled area, wherein the first ionic compound can release ions in the soil area to increase the electrical current.

Implementations of the system may include one or more of the following. The smart diaper can further include a substrate comprising a second material, wherein the second ionic compound is configured to release ions from the substrate into the soil area to increase the electrical current. The absorption pad can include more than 5 g/L of salt of the ionic compound in the soiled area. At least portions of the first electrode sensing circuit and the second electrode sensing circuit can be positioned parallel to each other. The absorption pad forms an absorption layer, wherein the first electrode sensing circuit and the second electrode sensing circuit relative to the absorption layer can be positioned side by side relative to the absorption layer. The absorption pad can form an absorption layer, wherein the first electrode sensing circuit can be positioned above the second electrode sensing circuit relative to the absorption layer. The controller can include a semiconductor chip in connection with the pair of electrode sensing circuits and configured to produce the voltage and to measure the electrical current that increases with the area of the soiled area; and an antenna that can transmit a wireless signal to alert a caregiver based on the electrical current measured by the semiconductor chip.

In another aspect, the present invention relates to a smart diaper to be worn by a user that includes a substrate comprising a first ionic compound; an absorption pad on the substrate and configured to absorb a liquid to form a soiled area; a pair of electrode sensing circuits comprising a first electrode sensing circuit and a second electrode sensing circuit, which are in contact with the absorbent material; and a controller that can apply a voltage across the pair of electrode sensing circuits and to produce an electrical current that increases with area of the soiled area, wherein the first ionic compound can release ions from the substrate into the soil area to increase the electrical current.

In yet another aspect, the present invention relates to a smart diaper to be worn by a user that includes an absorption pad that can absorb a liquid to form a soiled area; a thin film in contact with the absorption pad, wherein the thin film comprises an ionic compound; a pair of electrode sensing circuits comprising a first electrode sensing circuit and a second electrode sensing circuit, which can be in contact with the absorbent material; and a controller configured to apply a voltage across the pair of electrode sensing circuits and to produce an electrical current that increases with area of the soiled area, wherein the thin film can release ions into the soil area to increase the electrical current.

These and other aspects, their implementations and other features are described in detail in the drawings, the description and the claims.

### BRIEF DESCRIPTION OF THE DRAWINGS

FIG. 1 illustrates a smart diaper in accordance with some embodiments of the present application.

FIGS. 2A and 2B are respectively a top view and a cross-sectional view of a portion of a smart diaper having a pre-treated absorbent material in accordance with some embodiments of the present application.

FIGS. 3A and 3B are respectively a top view and a cross-sectional view of a portion of a smart diaper comprising a pre-treated substrate in accordance with some embodiments of the present application.

FIGS. 4A and 4B are respectively a top view and a cross-sectional view of a portion of a smart diaper comprising a pre-treated thin film in accordance with some embodiments of the present application.

### DETAILED DESCRIPTION OF THE INVENTION

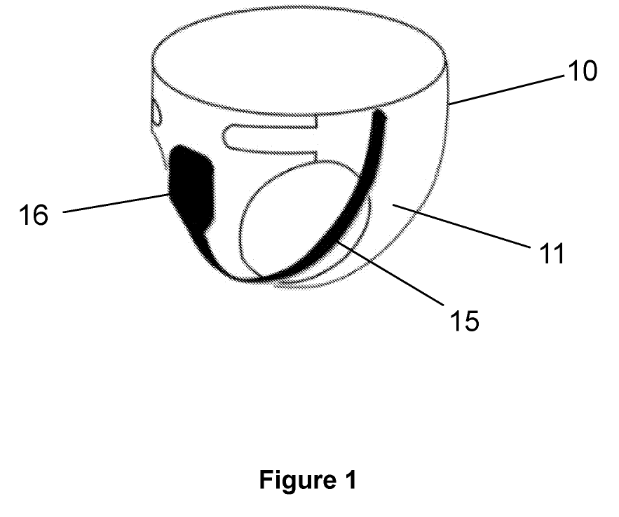

Referring to FIG. 1, a smart diaper 10 includes an absorption material 11, sensing electrodes 15, and a controller 16. The controller 16 also includes a semiconductor chip configured to process measurement signals, a battery configured to power the sensing electrodes 15 and the semiconductor chip, an antenna configured to wirelessly transfer data to an external device, and other electronic components. For example, the semiconductor chip can produce a voltage applied across sensing electrodes 15 and can measure an electrical current across the sensing electrodes 15. The wirelessly transferred data can include wetting status, and/or the degree of wetting or the size of the wetting area in the smart diaper 10.

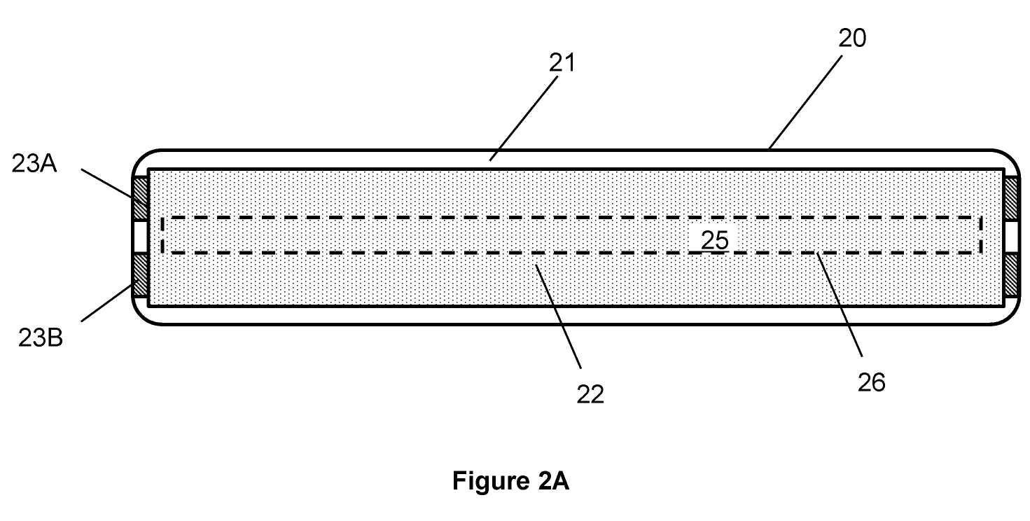

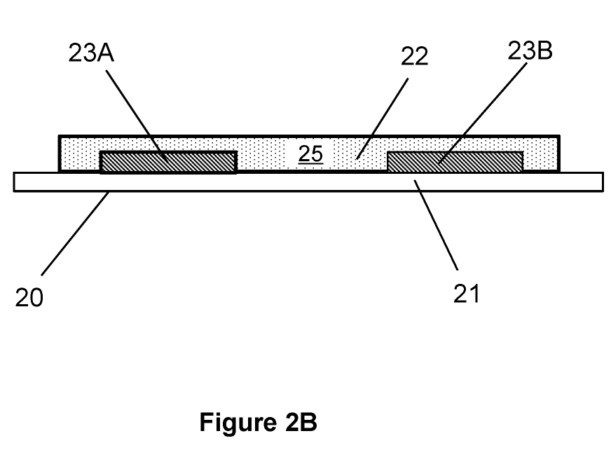

In some embodiments, referring to FIGS. 2A and 2B, a smart diaper 20 includes a substrate 21, an absorbent pad 22, and a pair of electrode sensing circuits 23A and 23B that are in contact with or embedded in the absorbent pad 22. At least portions of electrode sensing circuits 23A and 23B parallel to each other.

The absorption pad 22 is specially designed to absorb and retain body fluids. For example, the absorption pad 22 can include a nonwoven fabric that gives the smart diaper 20 a comfortable shape and helps prevent leakage. In another example, the absorbent pad 22 can be composed of a hydrophilic polymer and a fibrous material such as wood pulp. The polymer can be made of fine particles of an acrylic acid derivative. These polymeric particles act as tiny sponges that retain many times their weight in water.

The smart diaper 20 can be made in a multi-step process in which an absorbent pad is first vacuum-formed, the electrode sensing circuits 23A and 23B are embedded in or attached to the absorbent pad. Then the absorbent pad with the electrode sensing circuits 23A and 23B is attached to a permeable top sheet and an impermeable bottom sheet. The components are sealed together by application of heat or ultrasonic vibrations. When properly fitted, the smart diaper can retain body fluids, which pass through the permeable top sheet and are absorbed into the absorption pad 22 in the absorbent pad and sealed inside the impermeable bottom sheet.

In some embodiments, the absorbent pad 22 is pre-treated with an ionic compound 25 that is soluble in urine and form ions. For example, the absorbent pad 22 can be dosed with a water-soluble salt during the manufacture process. The water-soluble salt can be inorganic or organic, such as, sodium chloride, sodium acetate. The water-soluble salt can be pre-mixed with raw material of polymer or fibrous material in the absorbent pad 22. The absorbent pad 22 retains certain amount of solid ionic compound after it is dried.

When the smart diaper 20 is worn by a user and the absorbent pad 22 is wet by urine, at least a portion of the ionic compound 25 in the absorbent pad 22 is dissolved in the urine and release dissolved ions into the liquid, which can significantly increase electrical conductance (and decrease electrical resistance) between the electrode sensing circuits 23A and 23B.

Urine is an aqueous solution of greater than 95% water and with small amount sodium chloride and potassium chloride. Depending on the degree of wetting, pure urine itself often does decrease electrical resistance enough to be detected. In the presently disclosed smart diaper 20, ions dissolved in the liquid can significantly decrease the electrical resistance in the wet diaper, which is beneficial because it increases the signal-to-noise ratio and enhances detection sensitivity. When electrical voltage is applied across the electrode sensing circuits 23A and 23B, the measured electrical current between increases with the ion concentration. If pure urine contains salt at 1-2 g/L, sensing signal will change from person to person. If added ionic compound has an amount that is 5 times or ten times higher, measured conductance will be much less sensitive to human variation, and the measured signals will be much more stable. For example, the ionic compound added may result in more than 5 g/L of salt of the ionic compound in urine of the soiled area. In another example, the ionic compound added may result in more than 10 g/L of salt of the ionic compound in urine of the soiled area.

In some embodiments, a solution of the ionic compound 25 can be are disposed by a fluid delivery head (similar to an inkjet print head) in a spatial pattern in the absorbent pad 22. The ionic compound 25 can be placed in areas (e.g. 26) between the electrode sensing circuits 23A and 23B, and/or in the most commonly soiled area. The spatially patterned placement of the ionic material 25 can increase detection sensitivity in the most frequently wet areas of the smart diaper, but also reduce the usage of the ionic compound.

In some embodiments, the ionic compounds can be applied in conjunction with spatially distributed electrode sensing circuits. More details about the spatially distributed electrode sensing circuits are disclosed in commonly assigned U.S. patent application Ser. No. 15/889,375 “Smart diaper capable of sensing the size of a soiled area” filed Feb. 6, 2018, the disclosure of which is incorporated herein by reference.

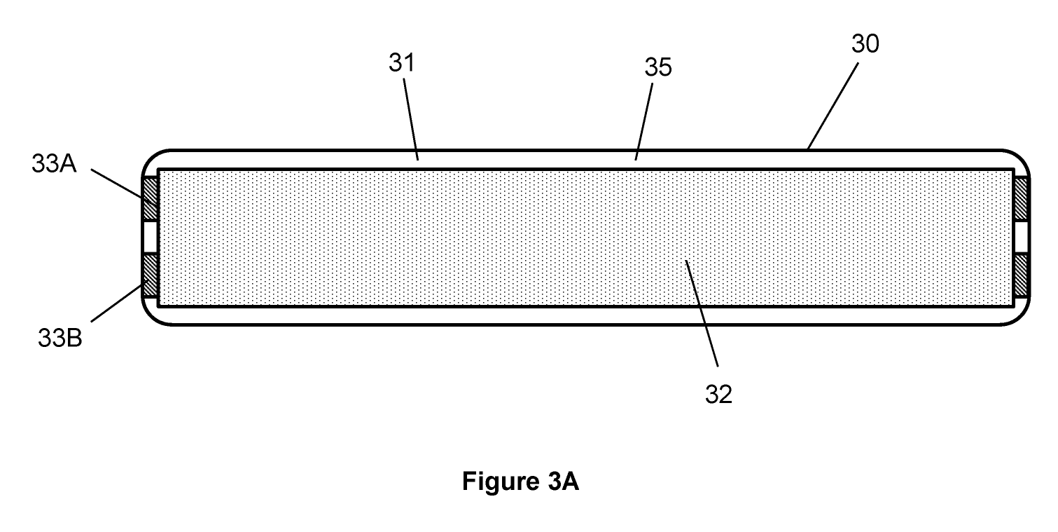

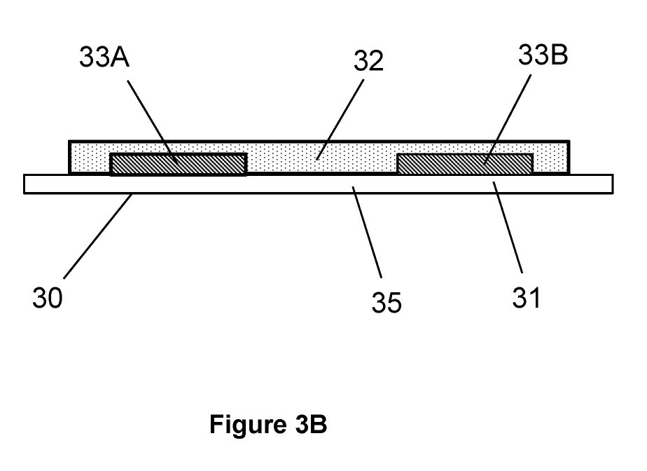

In some embodiments, the substrate material can be pre-treated with an ionic compound to enhance electrical conductance in the wet portion of a smart diaper. Referring to FIGS. 3A and 3B, a smart diaper 30 includes a substrate 31, an absorbent pad 32, and a pair of electrode sensing circuits 33A and 33B that are in contact with or embedded in the absorbent pad 32. At least portions of electrode sensing circuits 33A and 33B parallel to each other.

At least a portion of the substrate 31 is pre-treated with an ionic compound 35. For example, the substrate 31 can be dosed with a water-soluble salt during the manufacture process. The water-soluble salt can be inorganic or organic, such as, sodium chloride, sodium acetate. The water-soluble salt can also be pre-mixed with raw material of polymer or fibrous material in the absorbent pad 32. The substrate 31 retains certain amount of solid ionic compound after it is dried.

When the smart diaper 30 is worn by a user and the absorbent pad 32 is wet by urine, at least a portion of the ionic compound 35 in the substrate 31 is dissolved in the urine and release dissolved ions into the liquid in the absorbent pad 32, which can significantly increase electrical conductance (and decrease electrical resistance) between the electrode sensing circuits 33A and 33B. The ionic compound added in the substrate 31 may result in more than 5 g/L of salt of the ionic compound in urine of the soiled area. In another example, the ionic compound added in the substrate 31 may result in more than 10 g/L of salt of the ionic compound in urine of the soiled area.

In the presently disclosed smart diaper 30, ions dissolved in the liquid can significantly decrease the electrical resistance in the wet diaper, which is beneficial because it increases the signal-to-noise ratio and enhances detection sensitivity. When electrical voltage is applied across the electrode sensing circuits 33A and 33B, the measured electrical current between increases with the ion concentration.

An advantage of applying ionic compound 35 in the substrate 30 is that it provides flexibility to manufacturing process. For example, referring to FIG. 3B, the ionic compound 35 can be disposed in solution form by a liquid delivery device (similar to an inkjet print head) and absorbed by the substrate 31. The disposal can be in a first spatial pattern. Then the electrode sensing circuits 33A and 33B can be printed by delivering a conductive paste in a second spatial pattern. The two spatial patterns can be correlated with each other. For example, the ionic compound 35 can be placed along the adjacent areas of each of the electrode sensing circuits 33A and 33B.

In another example, the substrate 31 can be made of nonwoven fabrics. During nonwoven fabrics formation process, a certain amount of salts can be pre-mixed with raw material of plastic resins before the nonwoven fabrics are formed.

Another advantage of applying ionic compound 35 in the substrate 30 is that it can control the timing of the ionic release when the diaper is wet. Since the absorbent pad 32 is the component that absorbs the liquid first. Ions are only released from the substrate 35 after the liquid soiling has penetrated the depth of the absorbent pad 32. In other words, the absorbent pad 32 has exhausted its liquid retaining power and the smart diaper is in urgent need to be replaced.

It should be understood that the ionic compound 35 incorporated in the substrate 31 can work in conjunction with ionic compound (25 in FIGS. 2A and 2B) in the absorbent pad (22, or 32) to achieve optimal effects in the timing and the locations of the ionic releases.

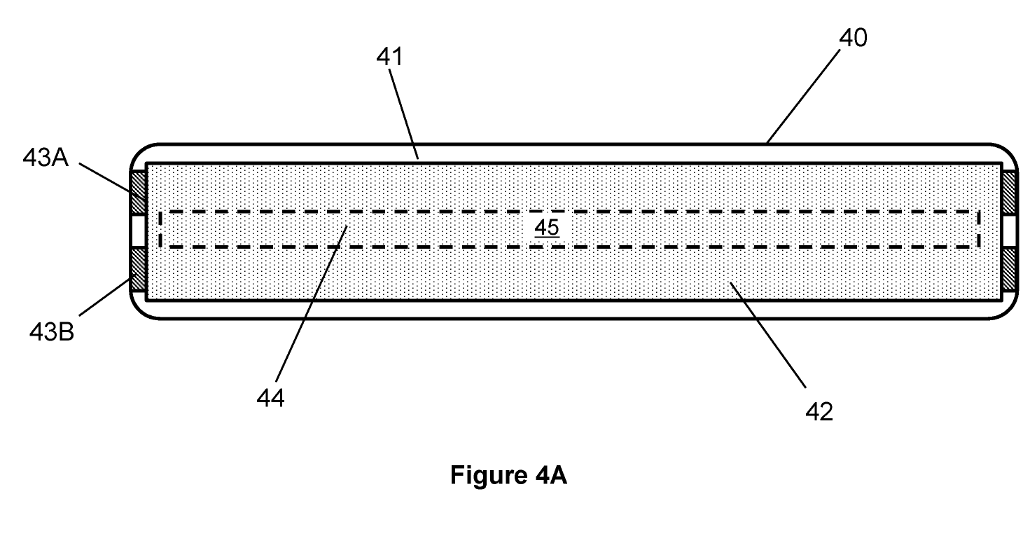

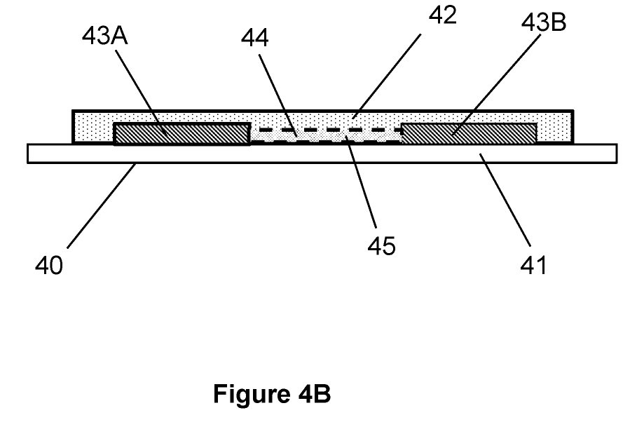

In some embodiments, referring to FIGS. 4A and 4B, a smart diaper 40 includes a substrate 41, an absorbent pad 42, and a pair of electrode sensing circuits 43A and 43B that are in contact with or embedded in the absorbent pad 42. At least portions of electrode sensing circuits 43A and 43B parallel to each other. The smart diaper 40 also includes a thin film 45 that can be placed on the substrate 41, between or on the two electrode sensing circuits 43A and 43B. The thin film 44 contains ionic compound 45 such as water soluble salts, which can release ions when the thin film 44 absorbs urine.

When the smart diaper 40 is worn by a user and the absorbent pad 42 is wet by urine, at least a portion of the ionic compound 45 in the thin film 44 is dissolved in the urine and release dissolved ions into the liquid in the absorbent pad 42, which can significantly increase electrical conductance (and decrease electrical resistance) between the electrode sensing circuits 43A and 43B. The ions dissolved in the liquid can significantly decrease the electrical resistance in the wet diaper, which is beneficial because it increases the signal-to-noise ratio and enhances detection sensitivity. When electrical voltage is applied across the electrode sensing circuits 43A and 43B, the measured electrical current between increases with the ion concentration. The ionic compound added in the thin film 44 may result in more than 5 g/L of salt of the ionic compound in urine of the soiled area. In another example, the ionic compound added in the thin film 44 may result in more than 10 g/L of salt of the ionic compound in urine of the soiled area.

An advantage for the thin film 44 comprising the ionic compound 45 is that it provides flexibility to manufacturing process. The thin film 44 can be separately made and treated from the substrate 41 and the absorption pad 42. Moreover, the thin film 44 can be made in a desirable spatial pattern to cover the most frequently wet area of the smart diaper 40. Furthermore, the amount ionic compound and the thickness of the thin film 44 can be varied to achieve the effect of enhancing electrical conductance when the smart diaper is soiled.

Another advantage of the pre-treated thin film 44 is that it can control the timing of the ionic release when the diaper is wet. Since the absorbent pad 42 is the component that absorbs the liquid first. Ions are only released from the thin film 44 after the liquid soiling has penetrated the depth of the absorbent pad 42. In other words, the absorbent pad 32 has exhausted its liquid retaining power and the smart diaper is in urgent need to be replaced. It should be understood that the pre-treated thin film 44 can work in conjunction with ionic compound (25 in FIGS. 2A and 2B, and 35 in FIGS. 3A and 3B) in the absorbent pad and the substrate to achieve optimal effects in the timing and the locations of the ionic releases.

It should be noticed that the electrode sensing circuits can be positioned in other configurations such as side by side on the substrate, as described above, or one above the other, etc. The electrode sensing circuits can include straight portions, and curved portions. Other spatial distributions of the electrode sensing circuits are disclosed in commonly assigned U.S. patent application Ser. No. 15/889,375 “Smart diaper capable of sensing the size of a soiled area” filed Feb. 6, 2018, the disclosure of which is incorporated herein by reference.

While this document contains many specifics, these should not be construed as limitations on the scope of an invention that is claimed or of what may be claimed, but rather as descriptions of features specific to particular embodiments. Certain features that are described in this document in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or a variation of a sub-combination.

Only a few examples and implementations are described. Other implementations, variations, modifications and enhancements to the described examples and implementations may be made without deviating from the spirit of the present invention.

What is claimed is:

1. A smart diaper to be worn by a user, comprising:
an absorption pad configured to absorb a liquid to form a soiled area;
a first ionic compound in the absorption pad;
a pair of electrode sensing circuits comprising a first electrode sensing circuit and a second electrode sensing circuit, which are in contact with the absorbent material; and
a controller configured to apply a voltage across the pair of electrode sensing circuits and to produce an electrical current that increases with area of the soiled area, wherein the first ionic compound is configured to release ions in the soil area to increase the electrical current.

2. The smart diaper of claim 1, further comprising:
a substrate comprising a second material, wherein the second ionic compound is configured to release ions from the substrate into the soil area to increase the electrical current.

3. The smart diaper of claim 1, wherein the absorption pad includes more than 5 g/L of salt of the ionic compound in the soiled area.

4. The smart diaper of claim 1, wherein at least portions of the first electrode sensing circuit and the second electrode sensing circuit are positioned parallel to each other.

5. The smart diaper of claim 1, wherein the absorption pad forms an absorption layer, wherein the first electrode sensing circuit and the second electrode sensing circuit relative to the absorption layer are positioned side by side relative to the absorption layer.

6. The smart diaper of claim 1, wherein the absorption pad forms an absorption layer, wherein the first electrode sensing circuit is positioned above the second electrode sensing circuit relative to the absorption layer.

7. The smart diaper of claim 1, wherein the controller comprises:
a semiconductor chip in connection with the pair of electrode sensing circuits and configured to produce the voltage and to measure the electrical current that increases with the area of the soiled area; and
an antenna configured to transmit a wireless signal to alert a caregiver based on the electrical current measured by the semiconductor chip.

8. A smart diaper to be worn by a user, comprising:
a substrate comprising a first ionic compound;
an absorption pad on the substrate and configured to absorb a liquid to form a soiled area;
a pair of electrode sensing circuits comprising a first electrode sensing circuit and a second electrode sensing circuit, which are in contact with the absorbent material; and
a controller configured to apply a voltage across the pair of electrode sensing circuits and to produce an electrical current that increases with area of the soiled area, wherein the first ionic compound is configured to release ions from the substrate into the soil area to increase the electrical current.

9. The smart diaper of claim 8, further comprising:
a second ionic compound in the absorption pad, wherein the second ionic compound is configured to release ions in the soil area to increase the electrical current.

10. The smart diaper of claim 8, wherein the substrate includes more than 5 g/L of salt of the ionic compound in the soiled area.

11. The smart diaper of claim 8, wherein at least portions of the first electrode sensing circuit and the second electrode sensing circuit are positioned parallel to each other.

12. The smart diaper of claim 8, wherein the absorption pad forms an absorption layer, wherein the first electrode sensing circuit and the second electrode sensing circuit relative to the absorption layer are positioned side by side relative to the absorption layer.

13. The smart diaper of claim 8, wherein the absorption pad forms an absorption layer, wherein the first electrode sensing circuit is positioned above the second electrode sensing circuit relative to the absorption layer.

14. The smart diaper of claim 8, wherein the controller comprises:
a semiconductor chip in connection with the pair of electrode sensing circuits and configured to produce the voltage and to measure the electrical current that increases with the area of the soiled area; and
an antenna configured to transmit a wireless signal to alert a caregiver based on the electrical current measured by the semiconductor chip.

15. A smart diaper to be worn by a user, comprising:
an absorption pad configured to absorb a liquid to form a soiled area;
a thin film in contact with the absorption pad, wherein the thin film comprises an ionic compound;
a pair of electrode sensing circuits comprising a first electrode sensing circuit and a second electrode sensing circuit, which are in contact with the absorbent material; and
a controller configured to apply a voltage across the pair of electrode sensing circuits and to produce an electrical current that increases with area of the soiled area, wherein the thin film is configured to release ions into the soil area to increase the electrical current.

16. The smart diaper of claim 15, wherein the thin film includes more than 5 g/L of salt of the ionic compound in the soiled area.

17. The smart diaper of claim 15, wherein at least portions of the first electrode sensing circuit and the second electrode sensing circuit are positioned parallel to each other.

18. The smart diaper of claim 15, wherein the absorption pad forms an absorption layer, wherein the first electrode sensing circuit and the second electrode sensing circuit relative to the absorption layer are positioned side by side relative to the absorption layer.

19. The smart diaper of claim 15, wherein the absorption pad forms an absorption layer, wherein the first electrode sensing circuit is positioned above the second electrode sensing circuit relative to the absorption layer.

20. The smart diaper of claim 15, wherein the controller comprises:
a semiconductor chip in connection with the pair of electrode sensing circuits and configured to produce the voltage and to measure the electrical current that increases with the area of the soiled area; and
an antenna configured to transmit a wireless signal to alert a caregiver based on the electrical current measured by the semiconductor chip.
